(19) United States
(12) Patent Application Publication
(10) Pub. No.: US 2026/0273121 A1
(43) Pub. Date: Sep. 17, 2026

(54) STERILIZATION CONTAINERS

(71) Applicant: Surgitray, LLC, Gulfport, FL (US)

(72) Inventors: Michele MAUZERALL, Gulfport, FL (US); Maryellen KEENAN, Gulfport, FL (US)

(73) Assignee: Surgitray, LLC, Gulfport, FL (US)

(21) Appl. No.: 19/078,074

(22) Filed: Mar. 12, 2025

Related U.S. Application Data
(63) Continuation of application No. PCT/US2025/019582, filed on Mar. 12, 2025.
(60) Provisional application No. 63/564,456, filed on Mar. 12, 2024.

(51) Int. Cl. A61L 2/26

(52) U.S. Cl. CPC A61L 2/26; A61L 2202/122

(57) ABSTRACT

Sterilization containers for sterilization of instruments such as surgical instruments implants, or the like. The containers have a filter that forms a portion of a wall of the container, where the filter is sufficiently resilient and/or non-deformable under ordinary handling of the container preventing the need for disposable filters. The containers have a lower section and a cover portion that is configured to sealingly join with the lower portion to maintain a sterility of an interior of the container after a sterilization process.

### CROSS-REFERENCE TO RELATED APPLICATIONS

This application is a continuation of PCT/US2025/019582 filed Mar. 12, 2025, which is a non-provisional of U.S. Provisional Application No. 63/564,456 filed Mar. 12, 2024, the entirety of which is incorporated by reference.

### FIELD OF THE INVENTION

Sterilization containers for sterilization of instruments such as surgical instruments implants, or the like. The containers have a filter that forms a portion of a wall of the container, where the filter is sufficiently resilient and/or non-deformable under ordinary handling of the container preventing the need for disposable filters. The containers have a lower section and a cover portion that is configured to sealingly join with the lower portion to maintain a sterility of an interior of the container after a sterilization process.

### BACKGROUND OF THE INVENTION

Surgical tools, instruments, and/or implants must be sterilized prior to a medical or surgical procedure in order to avoid contaminants from entering the surgical field. Sterilizable containers are often used to sterilize such surgical components and then transport the items a desired location, such as an operating room. For example, U.S. Pat. No. 4,783,321 teaches the use of such containers with a vented area that requires disposable paper filters that are sealed over the vent. Additional examples of sterilizable containers can be found in the following patents and publications: U.S. Pat. Nos. 4,331,257, 5,202,098, 6,217,835, 6,319,481, 7,001,441, 7,172,740, 9,381,263, 10,939,975, 11,273,233, US20050238530, US20030118491, US20220241449, and US20230037088. The entirety of each of the patents and publications listed above are incorporated by reference.

Transporting the implements within a sterilized container allows maintaining the sterility of the implements so that they can be removed from the container in a sterile environment. Accordingly, the containers are often durable during ordinary handling of the container and reusable for subsequent sterilization processes. However, conventional sterilizable containers rely on natural or synthetic filters that are positioned adjacent to a vented area of the container. These filters often absorb condensation during the sterilization process, which is undesirable when the container is opened. The integrity of such filters is also easily compromised through ordinary handling and can result in the need to re-sterilize the container in those cases where the filter integrity is found to have been compromised. The sterilization containers described herein provide an improved container with integral filters that are usable with a sterilizing agent and/or an autoclave that sterilizes the component stored within the sealed container.

There remains a need for a sterilized container comprised of durable parts that act as filters and are resistant to compromised integrity as is common with disposable filters that are prone to wrinkles, holes, wear, tears, water absorption.

### BRIEF DESCRIPTION OF THE FIGURES

Variations of the present disclosure include a sterilization container for use in a sterilization process, the sterilization container including: a base structure including a plurality of walls that define an interior region; a filter material forming at least a portion of a wall of at least one of the plurality of walls, where the filter material is integral with the wall such that the filter material is not releasable from the wall and the filter material is configured provide a fluid path between an exterior of the sterilization container and the interior region, wherein the base structure includes a sealable configuration wherein passage of a sterilizing agent must pass through the filter material to enter the interior region.

Additional variations of sterilization containers can include a lid portion; a base structure, where the lid portion is sealingly joinable to the base structure to form an interior region configured to hold one or more medical implements; a rigid filter forming a filtered portion of a wall of the sterilization container, where the rigid filter is non-removably secured against an adjacent region of the wall and provides a fluid path between an exterior of the sterilization container to the interior region to allow passage of a sterilizing agent therethrough while preventing contaminants from passing therethrough.

Additional sterilization containers can include a lid portion; a base portion, where the lid portion is sealingly joinable to the base portion to form a container having an interior region configured to hold one or more medical implements; wherein at least a portion of the lid portion and/or base portion includes a porous wall that provides a fluid path from an exterior of the container to the interior region to allow passage of a sterilizing agent therethrough while preventing contaminants from passing therethrough.

Variations of the present disclosure include a sterilization container for use in a sterilization process, the sterilization container including: a base structure including a plurality of walls that define an interior compartment; wherein each of the plurality of walls is fabricated from a filter material including a plurality of non-linear paths extending through the filter material to prevent any microbes from entering the interior compartment while allowing fluid to pass the plurality of non-linear paths filter during the sterilization process.

Variations of the present disclosure include a sterilization container, wherein the filter material includes a plurality of non-linear paths extending through the filter material to prevent any microbes from entering the interior compartment while allowing the sterilizing agent to pass the plurality of non-linear paths during the sterilization process.

Variations of the present disclosure include a sterilization container, wherein the plurality of non-linear paths extending in a tortuous pattern.

Variations of the present disclosure include a sterilization container, wherein the filter material includes at least one linear path extending through the filter material having a cross-dimension of 0.8 microns or less to prevent any microbes from entering the interior region while allowing fluid to pass through the at least one linear path during the sterilization process.

Variations of the present disclosure include a sterilization container, wherein an area of the filter material is greater than an area of a non-filtered wall.

Variations of the present disclosure include a sterilization container, further including one or more additional filter materials integral with one or more additional walls from the plurality of walls.

Variations of the present disclosure include a sterilization container, wherein a total surface area of the one or more additional filter materials and the filter material are greater than a total exterior surface area of a plurality of non-filtered walls.

Variations of the present disclosure include a sterilization container, wherein the wall includes a mesh structure.

Variations of the present disclosure include a sterilization container, wherein a majority of the wall includes the mesh structure.

Variations of the present disclosure include a sterilization container, wherein the plurality of walls includes a top portion having a top filter material integral with the top portion.

Variations of the present disclosure include a sterilization container, wherein the plurality of walls includes a bottom portion having a bottom filter material integral with the bottom portion.

Variations of the present disclosure include a sterilization container, wherein the base structure includes one or more stacking features to permit stacking of one or more additional sterilization containers on the base structure.

Variations of the present disclosure include a sterilization container, wherein at least one of the plurality of walls includes a top portion configured to be manipulated relative to a remainder of the base portion to permit access into the interior compartment.

Variations of the present disclosure include a sterilization container, further including a sealing member located between the top portion and a remainder of the base portion.

Variations of the present disclosure include a sterilization container, wherein the filter material is positioned within a pocket in the wall.

Variations of the present disclosure include a sterilization container, wherein the filter material is fastened to the wall by a process selected from the group consisting of a press fit, a sealant, a chemical bond, molding, extrusion, or a fastener.

Variations of the present disclosure include a sterilization container, wherein each of the plurality of walls includes one or more filter materials.

Variations of the present disclosure include a sterilization container, wherein at least a portion of the base structure includes a structure that undergoes a visual change or a tactile change as a result of the sterilization process.

Variations of the present disclosure include a sterilization container, wherein an exterior of the base structure includes one or more channels to drain liquid from the base structure.

Variations of the present disclosure include a sterilization container, wherein a portion of the wall is exterior to the filter material on an exterior surface of the base structure.

Variations of the present disclosure include a sterilization container, wherein the filter material forms an exterior surface of the base structure.

Variations of the present disclosure include a sterilization container, wherein the filter material is rigid.

Variations of the present disclosure include a sterilization container, wherein the filter material is elastic or deformable.

Variations of the present disclosure include a sterilization container, wherein the rigid filter includes a plurality of non-linear paths extending through the rigid filter to prevent any microbes from entering the interior region while allowing the sterilizing agent to pass the plurality of non-linear paths during the sterilization process.

Variations of the present disclosure include a sterilization container, wherein the plurality of non-linear paths extend in a tortuous pattern.

Variations of the present disclosure include a sterilization container, wherein the rigid filter includes at least one linear path extending through the rigid filter having a cross-dimension of 0.8 microns or less to prevent any microbes from entering the interior region while allowing fluid to pass through the at least one linear path during the sterilization process.

Variations of the present disclosure include a sterilization container, wherein an area of the filtered portion of the wall is greater than an area of a non-filtered portion of the wall.

Variations of the present disclosure include a sterilization container, further including one or more additional rigid filters integral sterilization container.

Variations of the present disclosure include a sterilization container, wherein the wall includes a mesh structure.

Variations of the present disclosure include a sterilization container, wherein a majority of the wall includes the mesh structure.

Variations of the present disclosure include a sterilization container, wherein the lid portion includes a top rigid filter integral with the lid portion.

Variations of the present disclosure include a sterilization container, wherein the base structure includes a bottom portion having a bottom rigid filter integral with the bottom portion.

Variations of the present disclosure include a sterilization container, wherein the base structure includes one or more stacking features to permit stacking of one or more additional sterilization containers on the base structure.

Variations of the present disclosure include a sterilization container, wherein the lid portion is configured to be manipulated relative to a remainder of the base structure to permit access into the interior region.

Variations of the present disclosure include a sterilization container, further including a sealing member located between the lid portion and a remainder of the base structure.

Variations of the present disclosure include a sterilization container, wherein the rigid filter is positioned within a pocket in the wall.

Variations of the present disclosure include a sterilization container, wherein the rigid filter is fastened to the wall by a process selected from the group consisting of a press fit, a sealant, a chemical bond, molding, extrusion, or a fastener.

Variations of the present disclosure include a sterilization container, wherein each side of the base structure includes one or more rigid filters.

Variations of the present disclosure include a sterilization container, wherein at least a portion of the base structure includes a structure that undergoes a visual change or a tactile change as a result of the sterilization process.

Variations of the present disclosure include a sterilization container, wherein an exterior of the base structure includes one or more channels to drain liquid from the base structure.

Variations of the present disclosure include a sterilization container, wherein a portion of the wall is exterior to the rigid filter on an exterior surface of the base structure.

Variations of the present disclosure include a sterilization container, wherein the rigid filter forms an exterior surface of the base structure.

### BRIEF DESCRIPTION OF THE FIGURES

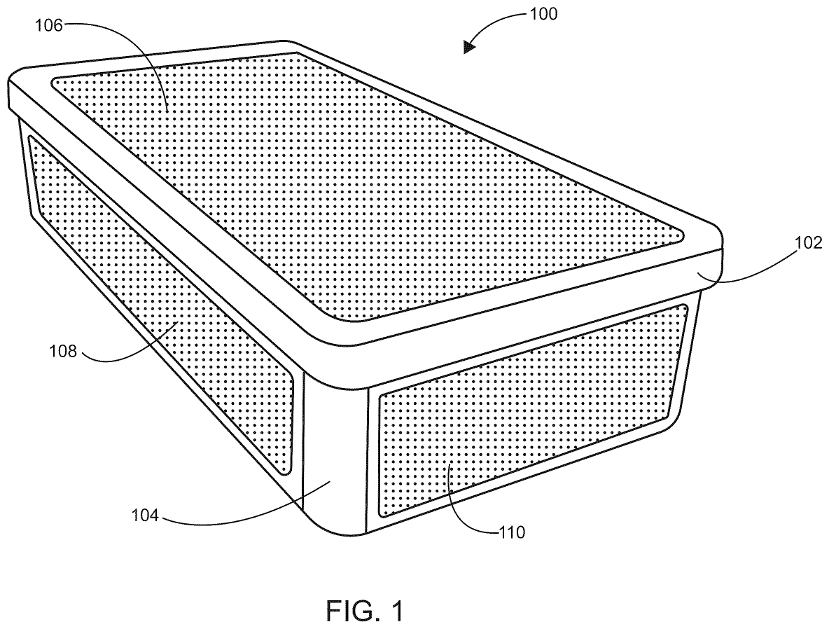

FIG. 1 illustrates a basic example of a sterilization container with integrated filters.

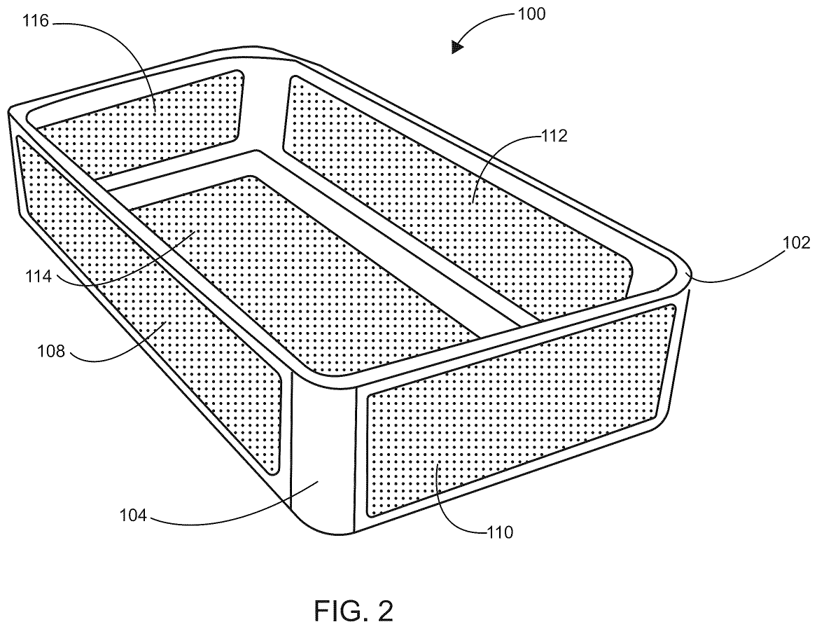

FIG. 2 illustrates a container as discussed in FIG. 1 with the lid removed to illustrate a portion of the interior compartment of the base.

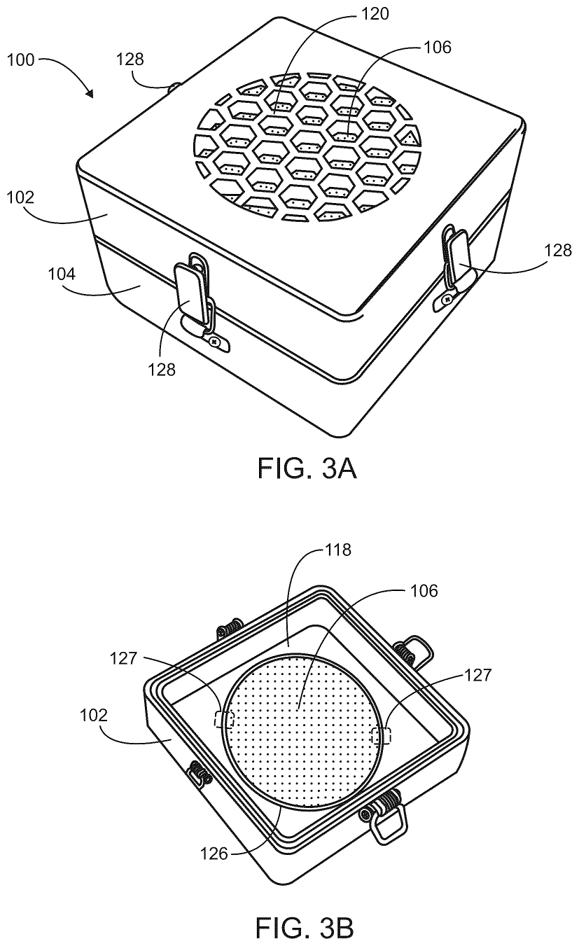

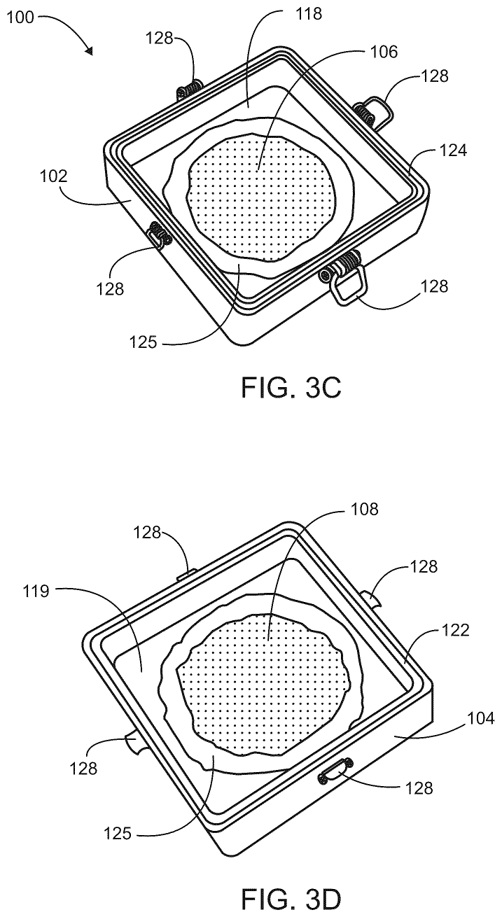

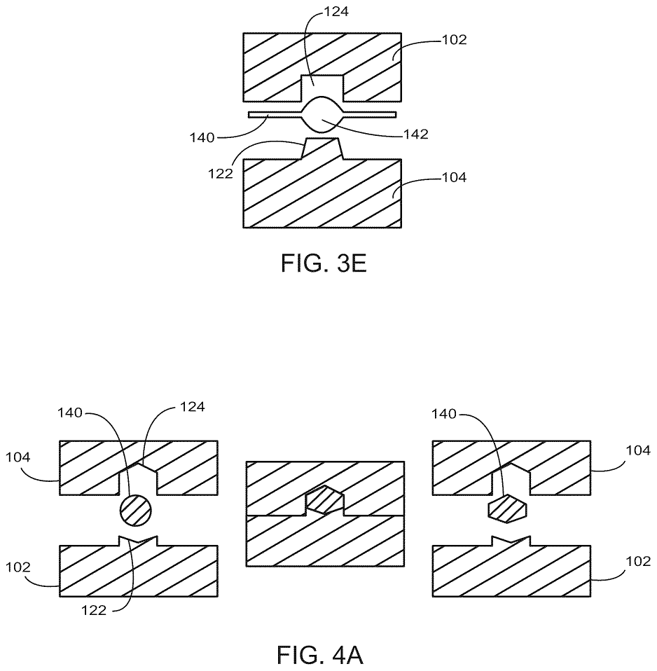

FIG. 3A illustrates another variation of a sterilization container having integrated durable filters.

FIGS. 3B, 3C and 3D show the container of FIG. 3A when the portions are separated.

FIG. 3E shows a partial cross-sectional view of a part the upper and lower portions to illustrate a gasket positioned between portions.

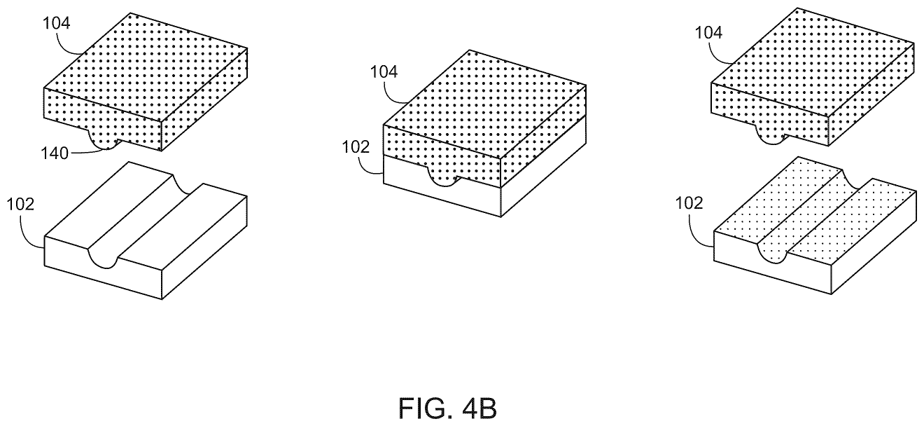

FIG. 4A shows a series of images to demonstrate deformation of a gasket to confirm that a proper seal is made during closing of the container.

FIG. 4B shows a visual or color coded seal.

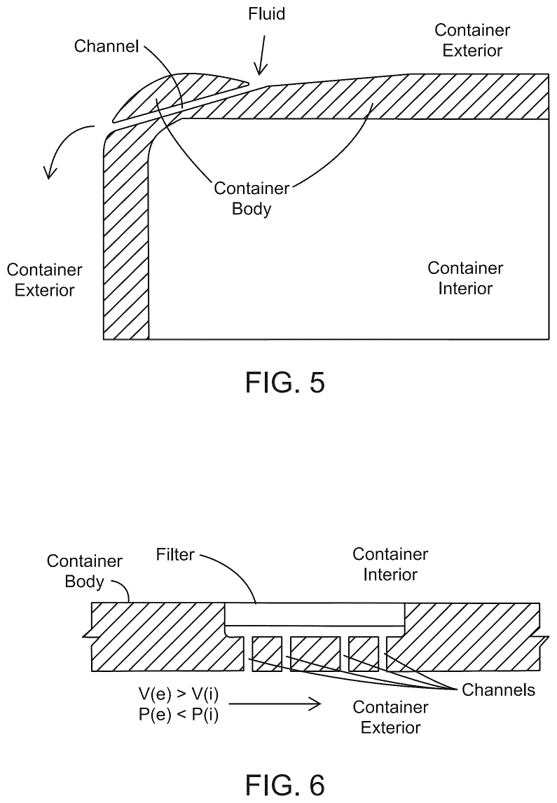

FIG. 5 illustrates the use of a channel, either internal or external, in the body of the container to direct steam, steam condensate, or other fluid.

FIG. 6 illustrates one or more internal channels in the body of the container to create a pressure differential that can drive/pull condensate out of the container via a Venturi effect.

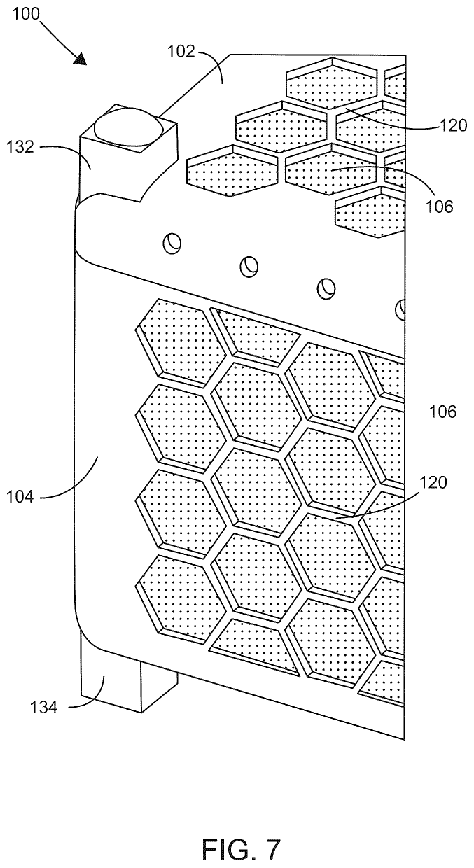

FIG. 7 illustrates another variation of channels that can direct the condensate (via gravity or vacuum) as well as a partial view of another variation of a container's 100 stacking features.

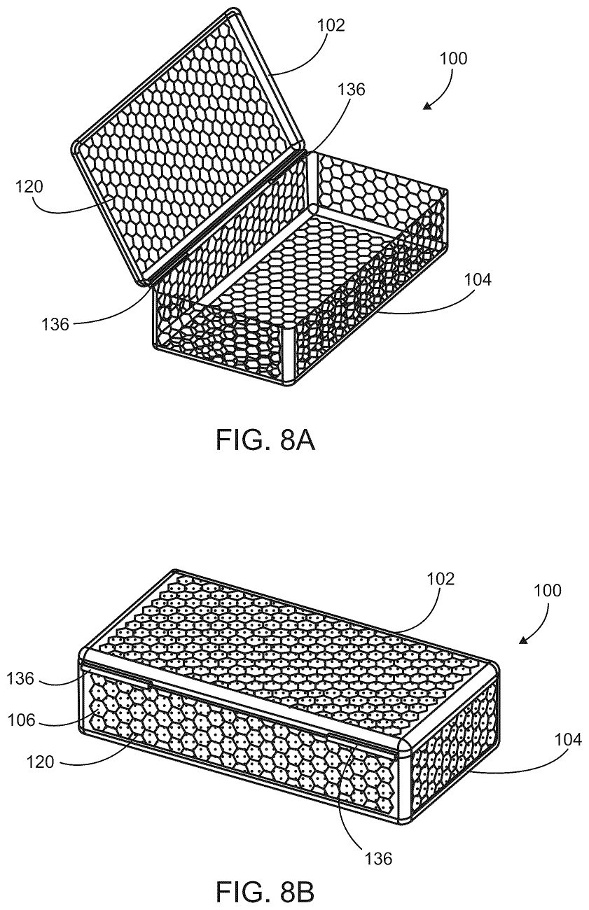

FIG. 8A illustrates a variation of a protective exoskeleton without any filtering structure for a container.

FIG. 8B shows the exoskeleton of FIG. 8A with a filter material forming part of the walls and a lid of the container.

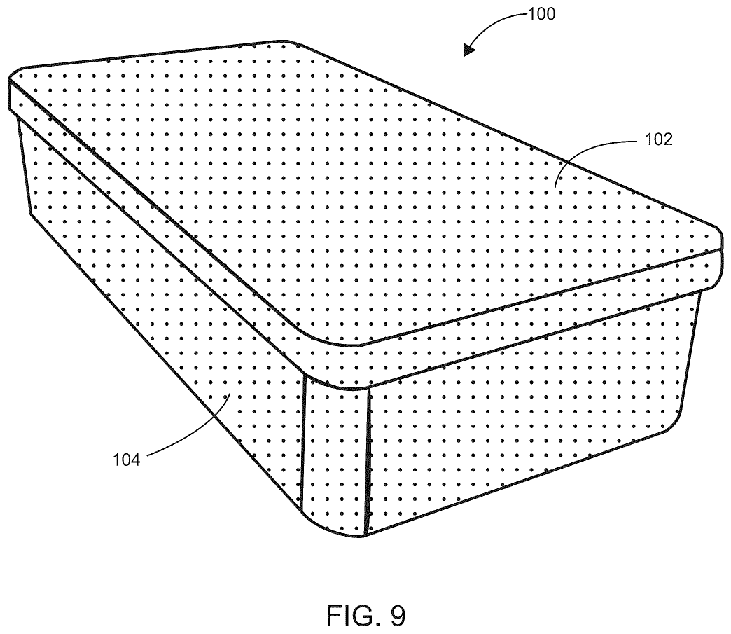

FIG. 9 shows a container in a closed configuration, where the container is fabricated from an engineered material that functions as a filter to permit movement of fluids through the wall/structure of the container.

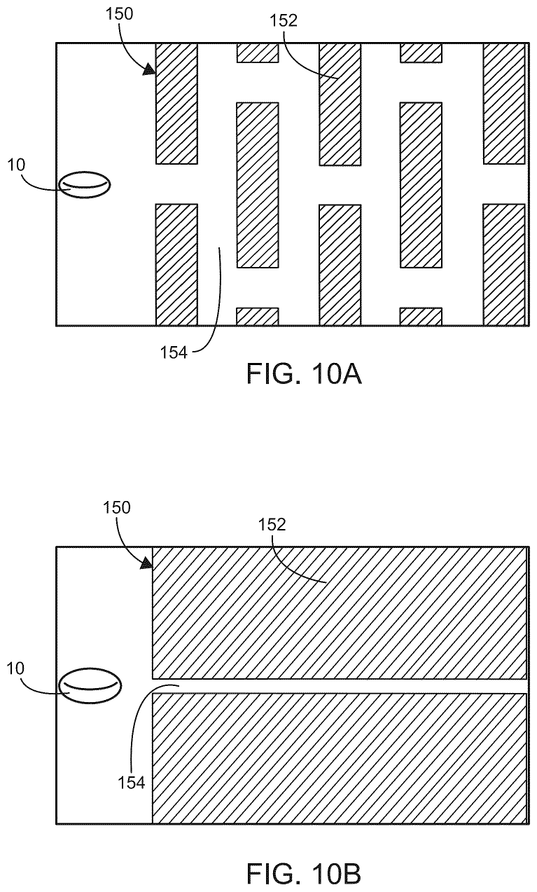

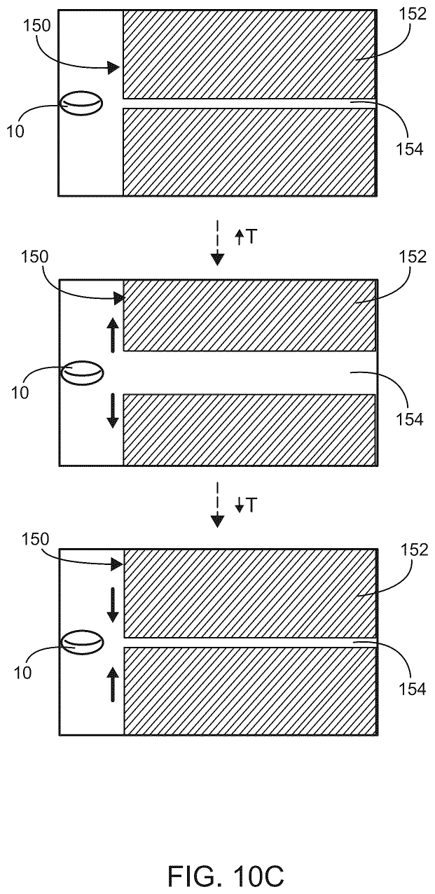

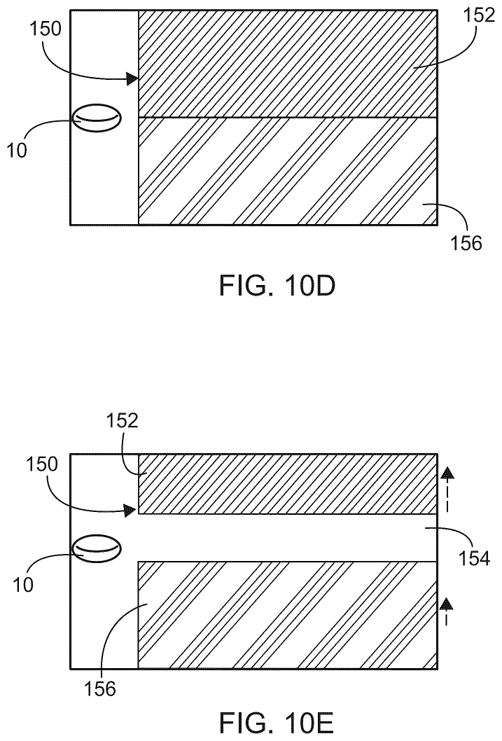

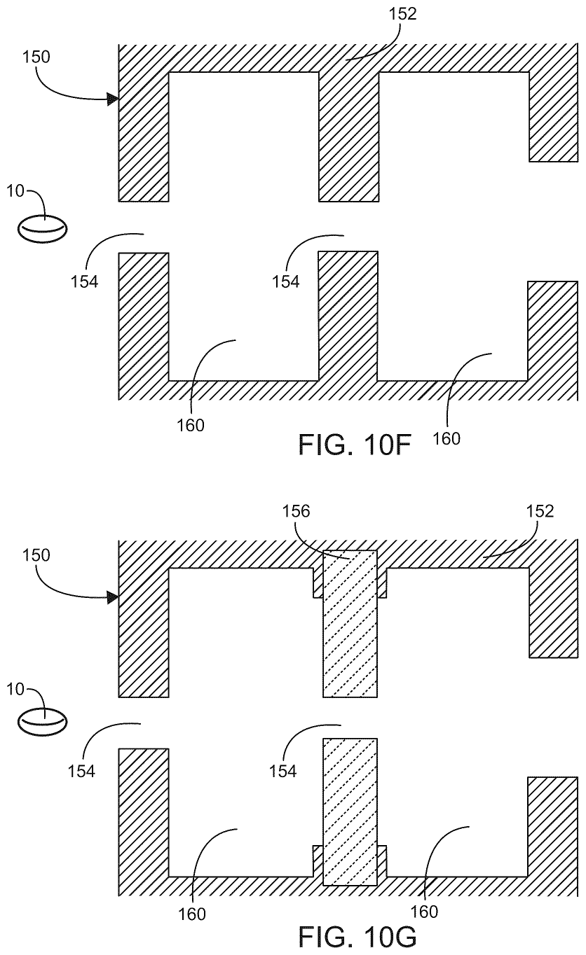

FIGS. 10A to 10G illustrate variations of a filter wall that functions as a porous filter and a wall structure for sterilization containers.

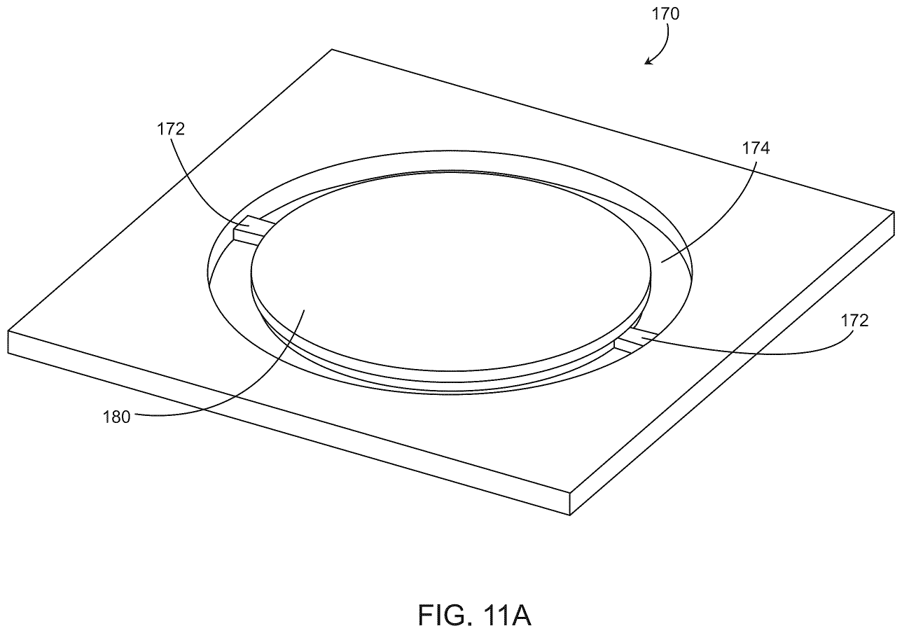

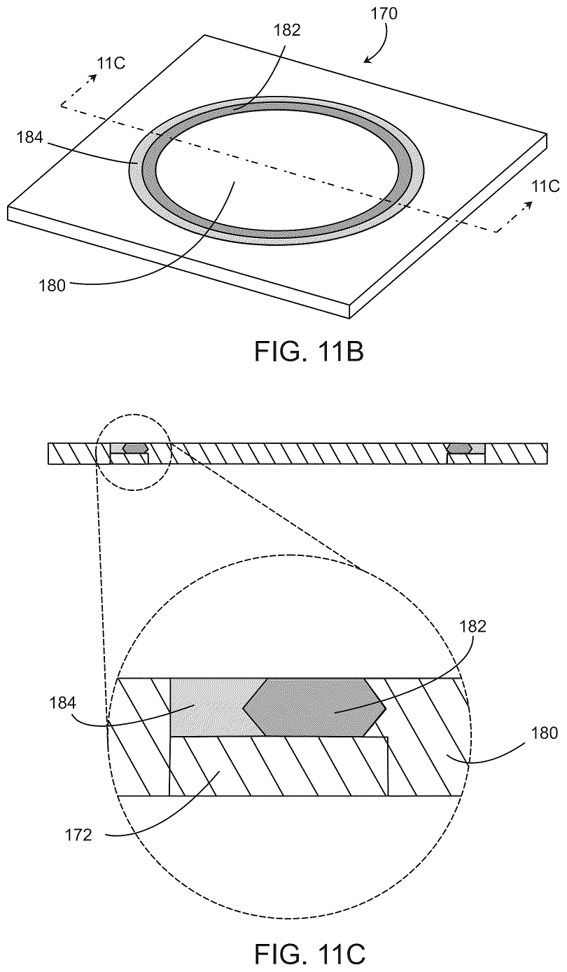

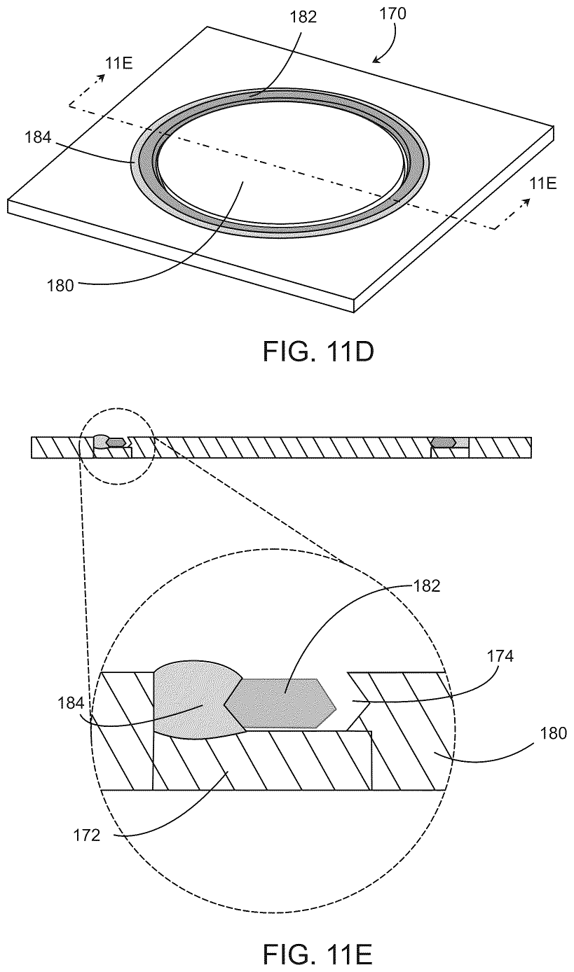

FIGS. 11A to 11E illustrate a variation of a thermal clutch design for a sterilization container.

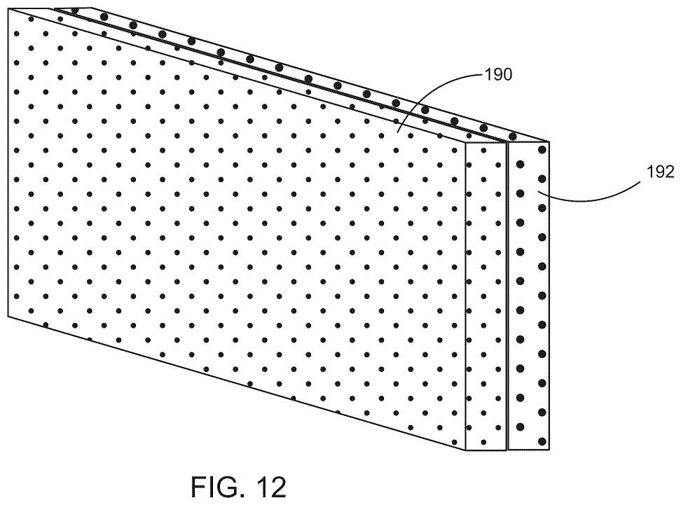

FIG. 12 shows an example where different filters are stacked.

### DETAILED DESCRIPTION

FIG. 1 illustrates a basic example of a sterilization container 100 with integrated filters 106, 108, 110. The container 100 can include a lid 102 that can be sealingly joined to a base 104, which comprises a non-filtered material. For purposes of illustration, additional features such as handles, closure mechanisms, information tag holders, etc. are not shown.

However, the containers 100 of the present disclosure can include any such features that are commonly used with sterilization containers, including but not limited to the components disclosed in the patents and publications listed above or those structures used with conventional sterilization equipment. As discussed below, the container 100 can use one or more seals or gaskets that are intermediate to the lid 102 and base 104 when joined to maintain an enclosure within the container that will prevent contaminants from entering the container 100. The containers described herein can have a filter region with a larger surface area than tradition/existing designs, such that the volume-to-vent (V-to-V) ratio is significantly decreased as compared to conventional containers. This shortens the dry time, which impacts efficiency. A shorter dry time is also safer for the patient than IUSS (immediate use steam sterilization), which allows for sterilized instruments that can be delivered in the same or less amount of processing time. The filter region is the region of the container that allows for passage of a sterilizing agent (including, but not limited to a vapor, a liquid, or any other sterilizing substance and/or cleaning substance) when the container is subjected to a sterilization process.

The containers 100 described herein can comprise any number of materials including but not limited to PLA, HIPS, PETG (PET/PETT), ABS, ASA, TPU (TPE/TPC/TPR), PP, OBC, PA 6/12/HTN, PMMA, PC, PET-CF, PA-CF, PS, PVDF, PSU, PES, PEI (9085/1010), TPI, PEI-CF, PPSU, DURATEM, PEEK, PEEK-CF, PEKK, PEKK-CF to enhance performance through physical and designed properties such as hydrophobic, antimicrobial, heat retention, heat deflection, stiffness, strength, hardness, corrosion resistance, tribology, faster drying time, weight, etc. The above listing of materials can include metals, glass, ceramics, etc.

In the illustrated example, the container 100 includes a filter region 106 as part of the lid 102 as well as multiple permeable filters regions 108, 110 on the base 104 that function to allow the sterilizing agent or other agent to pass therethrough while preventing contaminants from entering the container 100. The filters regions can comprise a ceramic material, porous metal, or a porous polymeric material that meets the structural requirements of the container 100. Namely, the filter materials are sufficiently durable such that they are not compromised during ordinary handling and usage of the container 100. This is in contrast to conventional filters typically made of materials susceptible to damage during ordinary handling. The filter materials can also comprise any material that creates a tortuous path for microbes and allows air, steam, condensate, and water to pass through. This tortuous path can include materials having various geometries to produce this effect. While variations of the filter regions can comprise rigid materials, the filter materials can comprise elastic or deformable materials that permit passage of the sterilizing agent or other agent while preventing contaminants from entering the container.

The container 100 discussed herein can be constructed using any process or method to integrate the filter with the body. Such processes or methods include, but are not limited to, press fit, spring-loaded retainer, clasp, elastic (compressible) retainer, tongue and groove, adhesive, chemical bond, over molding, direct extrusion, or fasteners. Where such fasteners can include, but are not limited to, screws, dowels, anchors, ball locks, quick-release fasteners, buttons, latches, rivets, nails, u-drive screws, nuts, clips, and bolts.

For example, the filters must be able to maintain a sealed interface with a remainder of the wall of the lid 102 and/or base 104 such that any fluid that enters the container 100 must pass through the porous filters. The durable filters can include materials that are non-rigid but have sufficient strength to maintain their function (e.g., porous, structural integrity, maintain a seal with the adjacent wall) in the container.

FIG. 2 illustrates a container 100 as discussed in FIG. 1 with the lid removed to illustrate a portion of the interior compartment of the base 104. As shown, the durable filters 108, 110, 112, 114, 116, 118 can be integral with the walls and the bottom of the base 104.

However, variations of containers 100 can include durable filters that are limited to certain areas of the container. Regardless of the construction, the filters provide a fluid path between an exterior of the container 100 and an interior of the container. In any case, fabrication of a container 100 with a durable filter that is integral to the construction of the container 100 permits a decreased V-to-V ratio (i.e., the ratio of the total volume of the container Vc to the surface area of the vented portion Vv of the container). This decreased V to V (for clarity “Vc-to-Vv”) ratio allows for a greater amount of sterilizing agent to quickly enter the container 100 allowing for a more thorough stabilization of the sterilant during the exposure phase as well as allowing for a faster dry time, which allows greater throughput in the sterilization department. The benefits include increased autoclave/equipment utilization, increased instrument turns/utilization, faster processing by the Sterilize Processing Department, shorter employee time to process since there are no filters to load/unload. The benefit of a low V-to-V ratio also allows for sterilization at IUSS speeds, but with AAMI/AORN full terminal sterilization parameters. Another key advantage of the improved containers having lower V-to-V ratio and durable reusable filters is the ability of the containers for use in rapidly cooling the contents without concern over condensation causing possible contamination.

In addition, unlike conventional sterilization containers, the integrated durable filters do not require protective grates or filter holders that require removal during application of a disposable filter. Furthermore, unlike traditional filters, the durable filters do not retain condensation produced as a result of the sterilization process and in some variations allow for rapid cooling relative to conventional containers.

The containers of the present disclosure can include a frame structure (e.g., the base 104 shown in FIG. 1 or FIG. 2) that is fabricated through any method such as stamping, molding, thermoforming, additive manufacturing, etc. and made of any material such as metal, glass, plastics, composites, rubbers, ceramics, resins, etc. as described above that holds the permanent, structural filters 106, 108, 110 in place. In an additional variation, the container 100 can be made entirely from the filter material (the filter could be made of plastics, metals, ceramics, rubbers, etc. In both cases, the container structure can have items attached to it, like screws, fasteners, gaskets, etc. In one variation of the container, the non-porous area of the container is minimized to maximize the filter region as much as possible.

In an additional variation, the containers can include a disposable toggle clamp that is either entirely disposable or include disposable components. The toggle clamp can also be lockable.

FIG. 3A illustrates another variation of a sterilization container 100 having a top portion 102 and a bottom portion 104 that can be sealingly joined together to provide a contaminant free interior for sterilization of implements located therein. In this variation, the top and bottom are for purposes of description since either portion can function as a lid and/or base. The container 100 also includes an integrated and durable filter 106 that is recessed within a wall of the top portion 102. This variation 100 also includes an optional protective mesh or grate 120 that covers the filter 106. In addition, the container 100 can include one or more fasteners 128 to secure the portions 102, 104 together. It is contemplated that the recessed feature of the filter 106 can be used in one or more of the integrated filters discussed above in FIGS. 1 and 2. FIG. 3B shows an underside of a portion 102 of the container of FIG. 3A to illustrate a durable filter 106 positioned in a pocket or space 126 within wall 118 of the portion 102, where the pocket 126 is adjacent to the mesh or grate 120 shown in FIG. 3A. The durable filter 106 can be press-fit, thermally fit, bonded, or otherwise joined to the wall 118 to provide a structure where any fluids or liquids entering or exiting the container 100. In additional variations, the durable filter 106 can be positioned against the wall 118 without a pocket. In an additional variation, the filters described herein can comprise non-durable or disposable filters in place of, or in combination with any containers described herein. For example, a container can include non-durable or disposable filters with any number of durable filters. Alternatively, a container can include all non-durable and/or disposable filters.

FIG. 3B also illustrates a variation where the filter 106 is retained against or within the wall 118 using one or more retainers/fasteners 127. Variations of the container allow for fasteners 127 that are irreversible or require tooling to release such that the filter 106 becomes a portion of the wall 118. In additional variations, the fasteners 127 can release the filter material 106 to permit removal of the filter 106 from the wall.

FIGS. 3C and 3D show the container of FIG. 3A when the portions 102, 104 are separated. As shown, the durable filters 106, 108 are integral with respective walls 118, 119 of portions 102, 104. As shown, durable filters 106, 108 are shown with an optional sealant 125 around the perimeter of the filters 106, 108 to maintain the filter integrally against or within the walls 118, 118 and to also maintain a barrier to contaminants. As noted above, securing the filter within or to the walls can be performed using any number of processes to provide a lasting seal.

FIGS. 3C and 3D also illustrate the portions 102, 104 as having a tongue 122 and groove 124 features to assist in formation of a seal between the portions 102, 104 as discussed below. FIG. 3E shows a partial cross-sectional view of a part the upper and lower portions 102, 104 to illustrate a gasket 140 positioned between portions 102, 104. The gasket 140 can optionally include a region of increased thickness 142 that is positioned within the groove 122 and further sealed when the tongue 124 is engaged against the increased thickness 142 of the gasket 140 when the portions 102, 104 are joined.

FIG. 4A shows a series of images to demonstrate a gasket 140 used to assist in sealing a first and second portions 102, 104. As shown, the tongue 122 and groove 124 can include shapes that deform the gasket 140 when properly seal. In the right-most image, the gasket 140 is deformed by the tongue 122 and groove 124, which indicates that a seal was made. In addition, or as an alternative, confirmation of a seal can occur by other means such as color, imprint, etc. For Example, FIG. 4B illustrates (from left to right) a gasket 140 integral with a second portion 104 (in alternate variations, the gasket can be separate) and a first portion 102. The first 102 and second 104 portions are then sealed together and then separated. However, the first portion 102 can include a structure that undergoes a visual change or other tactile change due to pressure or due to the sterilization process. The change can occur via a chemical reaction, mixing of media (e.g., wax or dyes), or by temperature/pressure sensitive paint.

FIG. 5 illustrates the use of a channel, either internal or external, in the body of the container to direct steam, steam condensate, or other fluid.

FIG. 6 illustrates one or more internal channels in the body of the container to create a pressure differential that can drive/pull condensate out of the container via a Venturi effect.

Any of the containers described herein can be made from a material or a combination of materials that interface directly, are adhered, or have intermediate interfacing material. In one variation, the containers are fabricated using additive manufacturing to create structures that have enhanced performance to be hydrophobic, antimicrobial, heat retention, heat deflection, stiffness, strength, hardness, corrosion resistance, tribology, faster drying time, etc. Examples are hollow or semi-hollow parts, meshes, multi-material parts, coatings, shields, reinforcement, etc. Moreover, the containers can be fabricated by using additive manufacturing and/or over molding to integrate the filter to the container body. Alternatively, the container can comprise a “pie tin” body with four side panels that connects to a bottom panel and a top panel.

The containers described herein can be used in any sterilization process. Including, but not limited to high-pressure steam, dry heat, chemical sterilants, and physical agents, such as pre-vacuum steam sterilization, gravity steam sterilization, dry heat, chemical sterilization, hydrogen peroxide, ethylene oxide, glutaraldehyde, hydrogen peroxide gas plasma, chlorine and chlorine compounds, ortho-phthalaldehyde, peracetic acid, formaldehyde, UV light, radiation, or any other sterilization process.

FIG. 7 illustrates a partial view of another variation of a container 100 having a small V-to-V ratio with filters 106 embedded within the container wall. In this variation a honeycomb structure 120 is positioned over the container. In addition, the container can include stacking elements 132, 134. The stacking elements 132, 134 can be positioned on a top 102 and bottom 104 of the container 100. The stacking features 132, 134 can be interlocking, nesting, etc. As shown, stacking feature 132 located on the top 102 can be designed to be inserted into a cavity on a receiving feature 134 to retain the stacked containers 100 together.

FIG. 8A illustrates a variation of a container 100 where the body is primarily formed to have a mesh or protective cover 120 over a majority of the surface. The container 100 includes a top portion 102 couple to a base portion 104 using a hinged construction. As shown, the container 100 can include one or more hinges 136. The hinge 136 can include a disposable hinge, disposable hinge components, a disposable locking rod/pin, a locking hinge, locking hinge components (like the rod as the disposable or permanent lock), disposable locking pin/rod. FIG. 8B shows the container 100 of FIG. 8A where the filter 120 is positioned against the grate or mesh 106 such that it provides a fluid path for a sterilizing fluid.

When the sterile containers are transported, stored, and delivered through unsterile environments, microbes must be prevented from entering the sterile interior of a container from these unsterile environments. Impermeable components of the container can prevent the entry of microbes. However, filters must allow fluids (such as steam, condensate, water, air, etc.) to pass through them at various stages in the sterilization cycle, while never allowing microbes to pass after the sterilization process has been completed.

The materials used for the filters discussed herein can comprise porous ceramics, which provide sufficient chemical stability, strength and rigidity, as well as thermal stability. One example of a porous ceramic is a ceramically bonded alumina provided by Filtros, LTD, East Rochester, NY. On one example, a Filtros, FAO-10, with a maximum pore diameter of 60 microns, was sufficient to provide a filter material for the containers disclosed herein.

Alternatively, or in combination, engineered materials can be provided to allow for fluid flow during a sterilization process but prevent microbes or other contaminants from entering the sterilized container after the sterilization process is completed.

As noted above, the various containers can include filter materials that form part of the wall. However, an additional variation of the containers includes a container 100 as shown in FIG. 9, where the container 100 is fabricated from an engineered material that functions as a filter to permit movement of fluids through the wall/structure of the container 100 but prevents passage of contaminants or microbes. The container 100 shown in FIG. 9 illustrates a lid 102 and base 104 formed from a filter material, where the locking, closing, hinge features are not shown. In some variations, traditional locking, closing, hinge features are used with the engineered filter material. In addition, less than the entirety of the container can be made from a filter material.

As noted above, various manufacturing processes can produce engineered materials to serve as a combination wall structure and filter (“filter wall”) with sufficient chemical stability, strength and rigidity, as well as thermal stability. Such filter walls can be used for any part of the containers disclosed herein, including but not limited to the walls, edges, corners, lids, etc.

The filter walls will have a fluid path that allows fluid flow during sterilization, but prevents microbe entry after sterilization. Such fluids paths can include: 1) a tortuous paths, 2) size restrictive paths that are smaller than the size of the microbe, or 3) fluid a path that is constructed of or includes antimicrobial material. Research has shown that a tortuous path can prevent the entry of microbes, even when the path is wider than the microbe.

FIG. 10A shows a partial cross-sectional view of an example of a filter wall 150 having a tortuous filter path 154 that prevents a microbe or other contaminant 10 from passing through the filter wall 150 from an exterior of a container. As shown, the filter wall material 152 is constructed to provide a tortuous or non-linear path 154 through the filter wall 150. The tortuosity of the path 154 prevents the microbe 10 from entering the container but still allows fluid flow during the sterilization process. The fluid path 154 can have a dimension that is larger than the microbe 10 since microbes generally lack locomotion, so they cannot traverse the tortuous path that is created by the filter wall material 152 in the filter wall 150. Methods such as sintering have been used to create a random tortuous path, but other manufacturing methods and materials (such as additive manufacturing) can be used to create intentional patterns of paths.

FIG. 10B shows a filter wall 150 having a filter wall material 152 with one or more linear fluid paths 154, where the size dimension of the fluid path 154 is smaller than the microbe to prevent passage of the microbe 10. This configuration can be achieved through designed voids created during additive manufacturing, or by reductive manufacturing using methods such as laser drilling and electrical discharge machining, etc. The size of the fluid path can be less than 0.8 micron in diameter/cross-dimension. However, this size can be greater in those configurations having a tortuous path.

FIG. 10C shows a representation of a filter wall 150 that structurally changes during a sterilization process. The top figure shows a filter wall 150 with a fluid pathway 154 that is smaller than a microbe at ambient temperature. Alternate variations include a fluid pathway 154 that is fully closed or occluded during ambient temperature. As shown in the middle figure, when the temperature increases, the filter wall material 152 can undergo a thermal expansion or compression that increases a dimension or size of the fluid pathway. The filter wall material 152 is designed to return to its initial configuration as the temperature decreases such that the prevent microbe entry.

FIGS. 10D and 10E illustrate yet another variation filter wall 150 comprising two or more materials 152, 156 that interface with each other. FIG. 10D represents the filter wall 150 at ambient temperature where the wall materials 152, 156 contact to occlude any fluid path. Alternatively, there can be some separation such that the fluid path is too small to allow migration of any microbe. It is noted that one or more materials 152, 156 The two materials 152, 156 have a different coefficient of thermal expansion (CTE). In illustration shown in FIG. 10E, as temperature increases, filter material 156 has a smaller CTE than filter material 152.

As temperature increases, material 152 expands more than material 156, creating a gap or fluid path 154 between the materials 152, 156. This fluid path allows fluid to pass during sterilization cycles. When the material cools, the gap returns to its initial condition preventing microbes from passing into the sterile contents.

FIGS. 10F and 10G show a variation of a fluid path 154 having chamber 160 including along the path 154 to prevent fluid from wicking microbes through the filter wall 150. The fluid paths 154 can be straight or tortuous. In these variations, the entry pathways 145 act as diffusers, and the chambers 160 act as surge tanks to reduce pressure gradients and fluid flow that could transport microbes 10 into the container. The fluid path 150 can have varying sizes along through the filter wall. FIG. 10F show a variation with homogenous material 154 forming the filter wall 150. FIG. 10G shows a variation with a different material 156 combined with a filter wall 152.

FIGS. 11A to 11D illustrate a variation of a thermal clutch design for a sterilization container. In these designs, as sterilization temperatures increase, a gap opens between two materials due to a difference in their coefficient of linear thermal expansion (CLTE, a). These structures are designed to remain tightly pressed against each other at ambient temperature, which prevents microbes from passing through the structure. As temperature increases during sterliztaion, a gaps open because of the different expansion properties of the materials. One material expands more than the other, allowing fluids such as steam, condensate, and water to pass through the gap.

FIG. 11A shows a panel 170 for use with any sterilization container. As shown, the panel 170 includes a gap 174 and a first material 180 adjacent to the gap 174. It is noted that the entire panel 170 can be fabricated from the first material 180, or the first material 180 can be limited to a portion of the panel 170 that is adjacent to the gap 174. Panel 170 also includes support members 172 as described below.

FIG. 11B shows the panel 170 under ambient conditions where a second material 182 and a third material 184 occlude the gap. In this variation, the first material 180 has a lower CLTE than the second material 180. Therefore, the first material 180 will expand less than the second material 182 given the same increase in temperature. The second material 182 has a higher CLTE and will therefore expand more than the first material 180 given the same increase in temperature.

FIG. 11C illustrates a cross-sectional view taken along line 11C=11C in FIG. 11B. As shown, the first material 180 is adjacent to the second material 180 to occlude the gap. A third material 184 is adjacent to the second material 182. This third material 184 can be a low durometer or expansion material that absorbs the difference in expansion between materials.

FIG. 11D represents a condition where the panel 170 is heated such that the second material 182 expands to a greater degree than the first material 180. As shown, the second material 182 can expand into a low durometer or expansion material 184. The difference in expansion allows the gap 182 to form a fluid path between an interior and exterior of the sterilization container as shown in FIG. 11E, which is a cross-sectional view taken along the line 11E-11E in FIG. 11D. It is noted that the panel can have one or more supports or structures on the exterior of the panel 170 to prevent the materials from being displaced out of the gap 174.

FIG. 12 shows a variation of a filter combination that can be used in any variation described herein. In this example, two filters 190, 192 are stacked together. These filters can be the same or different. For example, filter 190 can comprise a durable, non-durable, disposable filter, or a specific pore size, while filter 192 can comprise a same or different filter.

CLTE for each material is given in 10-6/° F. at ambient temperature. The higher the ratio, the larger the gap will be over the same temperature increase. Examples of materials and their CLTE are as listed below:
First material 180: Alumina (Al₂O3), 8.1; second material 182: Aluminum, 21-24; CLTE Ratio: 2.6-3.0
First material 180: Stainless Steel Austenitic (304), 17.3; second material 182: Polytetrafluorethylene (PTFE), 112-135; CLTE Ratio: 6.5-7.8
First material 180: Polypropylene, glass fiber-reinforced, 32; second material 182: Polypropylene, unfilled, 72-90; CLTE Ratio: 2.25-2.8
First material 180: Silicon, 3-5; second material 182: Polysulfone (PSO, PSU), 55-60; CLTE Ratio: 11.0-20.0

Any of the variations shown herein can be fabricated from antimicrobial materials or the materials can contain a coating or additive that provides antimicrobial properties.

As for other details of the present invention, materials and manufacturing techniques may be employed as within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described and equivalents (whether recited herein or not included for the sake of some brevity) may be substituted without departing from the true spirit and scope of the invention. Also, any optional feature of the inventive variations may be set forth and claimed independently, or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments, or the embodiments themselves can be combined. Where such combinations are intended to be within the scope of this disclosure.

1. A sterilization container for use in a sterilization process, the sterilization container comprising:
a lid;
a base structure formed into a mesh pattern over a majority of a surface of the sterilization container, where the base structure comprises a plurality of walls that define an interior region; and
a filter material positioned within the interior region and adjacent to the base structure such that the mesh pattern forms a protective cover over the filter material and the majority of the surface of the sterilization container, where the filter material is not releasable from the base structure and is configured provide a fluid path between an exterior of the sterilization container and the interior region, wherein the base structure and the lid include a sealable configuration wherein passage of a sterilizing agent must pass through the filter material to enter the interior region.

2. The sterilization container of claim 1, wherein the filter material comprises a plurality of non-linear paths extending through the filter material to prevent any microbes from entering the interior region while allowing the sterilizing agent to pass the plurality of non-linear paths during the sterilization process.

3. The sterilization container of claim 2, wherein the plurality of non-linear paths extending in a tortuous pattern.

4. The sterilization container of claim 1, wherein the filter material comprises at least one linear path extending through the filter material having a cross-dimension of 0.8 microns or less to prevent any microbes from entering the interior region while allowing fluid to pass through the at least one linear path during the sterilization process.

5. The sterilization container of claim 1, wherein an area of the filter material is greater than an area of a non-filtered wall.

6. The sterilization container of claim 1, further comprising one or more additional filter materials integral with one or more additional walls from the plurality of walls.

7. The sterilization container of claim 6, wherein a total surface area of the one or more additional filter materials and the filter material are greater than a total exterior surface area of a plurality of non-filtered walls.

8. (canceled)

9. (canceled)

10. The sterilization container of claim 1, wherein the lid includes a top filter material integral with the lid.

11. The sterilization container of claim 1, wherein the plurality of walls includes a bottom portion having a bottom filter material integral with the bottom portion.

12. The sterilization container of claim 1, wherein the base structure includes one or more stacking features to permit stacking of one or more additional sterilization containers on the base structure.

13. (canceled)

14. The sterilization container of claim 1, further comprising a sealing member located between the lid and the base structure.

15. (canceled)

16. The sterilization container of claim 1, wherein the filter material is fastened to the base structure by a process selected from the group consisting of a press fit, a sealant, a chemical bond, molding, extrusion, or a fastener.

17. The sterilization container of claim 1, wherein each of the plurality of walls includes one or more filter materials.

18. The sterilization container of claim 1, wherein at least a portion of the base structure includes a structure that undergoes a visual change or a tactile change as a result of the sterilization process.

19. The sterilization container of claim 1, wherein an exterior of the base structure includes one or more channels to drain liquid from the base structure.

20. (canceled)

21. The sterilization container of claim 1, wherein the filter material forms an exterior surface of the base structure.

22. The sterilization container of claim 1, wherein the filter material is rigid.

23. The sterilization container of claim 1, wherein the filter material is elastic or deformable.

24. (canceled)

25. (canceled)

26. A sterilization container for use in a sterilization process, the sterilization container comprising:
a lid; and
a base structure comprising a plurality of walls that define an interior region, the base structure is entirely formed from an engineered material configured to serve as a combination of a wall structure and a filter that provides a fluid path between an exterior of the sterilization container and the interior region, wherein the base structure and the lid include a sealable configuration wherein passage of a sterilizing agent must pass through the engineered material to enter the interior region.
